(19) United States
(12) Patent Application Publication
(10) Pub. No.: US 2023/0320728 A1
(43) Pub. Date: Oct. 12, 2023

(54) ENDOSCOPIC SURGICAL SYSTEM AND METHOD

(71) Applicant: Integrity Implants Inc., Palm Beach Gardens, FL (US)

(72) Inventors: Wyatt Drake Geist, 2621 Abiaca Circle, FL (US); Paul Houle, Palm Beach Gardens, FL (US); Raymond Gardocki, Palm Beach Gardens, FL (US); Caitlin Kealy, Palm Beach Gardens, FL (US)

(21) Appl. No.: 18/043,991

(22) PCT Filed: Sep. 3, 2021
(86) PCT No.: PCT/US21/49020

§ 371 Date: Mar. 3, 2023

Related U.S. Application Data
(60) Provisional application No. 63/074,797, filed on Sep. 4, 2020.

(51) Int. Cl. A61B 17/072; A61B 17/00

(52) U.S. Cl. CPC A61B 17/07207; A61B 1/00149; A61B 17/00234

(57) ABSTRACT

The invention involves a system and method for utilizing tools larger than the cannula bore of an endoscope in an endoscopic procedure. The system includes an endoscope having a cannula with an open or ported distal end. The ported distal end allows the shaft of an elongated tool having an oversized functional distal end to extend through the port with the oversized end in front of cannula bore. The tool and endoscope cannula are placed through a dilator tube that extends from outside the patient to the surgical site. A removeable and re-attachable handle is securable to the shaft of the oversized tool, which allows the surgeon to manipulate the tool through the cannula while visualizing the procedure utilizing the endoscope. This construction allows the surgeon to utilize tools that are too large to fit through the cannula of the endoscope including curettes, rasps, burrs, trials, Kerrison's, shavers and inserters.

### RELATED APPLICATIONS

This application is a national stage filing in accordance with 35 U.S.C. § 371 of PCT/US2021/049020, filed Sep. 3, 2021, which claims the benefit of the priority of U.S. Provisional Patent Application No. 63/074,797, entitled “ENDOSCOPIC SURGICAL SYSTEM AND METHOD”, filed on Sep. 4, 2020. The contents of each are incorporated herein by reference.

### FIELD OF INVENTION

The present invention generally relates to spinal surgery; and more particularly, to an endoscopic surgical system that provides for the use of surgical tools considered oversized for use with an endoscope.

### BACKGROUND INFORMATION

The spinal column, or backbone, is one of the most important parts of the body. It provides the main support, allowing us to stand upright, bend, and twist. As shown in FIG. 1, thirty-three (33) individual bones interlock with each other to form the spinal column. The vertebrae are numbered and divided into regions. The cervical vertebrae form the neck and allow nodding and shaking of the head. The thoracic vertebrae join with the ribs to form the rib cage. The five lumbar vertebrae carry most of the weight of the upper body and provide a stable center of gravity when a person moves. Five vertebrae of the sacrum and four of the coccyx are fused to comprise the back wall of the pelvis. Intervertebral discs are located between each of the mobile vertebra. Intervertebral discs comprise a thick outer layer with a crisscrossing fibrous structure annulus that surrounds a soft gel-like center, the nucleus. Discs function like shock-absorbing springs. The annulus pulls the vertebral bodies together against the elastic resistance of the gel-filled nucleus. When we bend, the nucleus acts like a ball bearing, allowing the vertebral bodies to roll over the incompressible gel. Each disc works in concert with two facet joints, forming a spinal motion segment. The biomechanical function of each pair of facet joints is to guide and limit the movement of the spinal motion segment. The surfaces of the joint are coated with cartilage that helps each joint move smoothly. Directly behind the discs, the ring-like vertebral bodies create a vertical tunnel called the spinal canal or neuro canal. The spinal cord and spinal nerves pass through the spinal canal, which protects them from injury. The spinal cord is the major column of nerve tissue that is connected to the brain and serves as a neural pathway between the brain and the body. The nerves in the spinal cord branch off to form pairs of nerve roots that travel through the small openings between the vertebrae and the intervertebral foramens.

Various medical conditions require a surgeon to repair, remove and/or replace injured or otherwise damaged or compromised discs. This surgical procedure is known as a discectomy (or diskectomy). During a discectomy, the surgeon removes the nucleus of the disk and replaces it with an implant.

One common tool used in these spinal surgical procedures is an endoscope. Endoscopes are complex biomedical devices through which a surgeon may visualize as well as perform portions of the surgical procedure. The complexity results from the need for fiberoptic bundles and multiple long narrow channels to be contained within a tubular structure that is constrained by the limited dimensions of the body cavity opening through which the surgery is performed.

Drawbacks associated with endoscopes include the limited size of the tube or channel through which the surgeon has available to perform the necessary work. Examples of endoscopes include U.S. Pat. No. 5,772,578 to Heimberger, el al. disclosing such a device which, while flexible, allows only one small piece of disc to be removed at a time using a grasping tool with alligator like jaws. This construction requires the surgeon to make one grasp at the tissue and remove the tool from the endoscope to clear the tissue before replacing the tool through the endoscope to make another grasp. This piece-meal removal is laborious and time consuming.

U.S. Pat. No. 8,109,957 to Stad et al. discloses a disc nucleus removal device with cutting members in its side wall near the tip deployed by virtue of slits in the side wall sheath. Since the cutting members are actuated from the side wall of the elongate member, the blunt tip impedes the ability of the device to cut tissue directly in front of it, and hence is better suited to end plate preparation than disc removal.

U.S. Patent Publication No. 2018/0028211 to Suddaby discloses an endoscope that includes an auger in the shaft portion of the endoscope for evacuating pieces of the disc. Since the auger takes so much space through the shaft of the endoscope, the Suddaby device is limited to a balloon that can be inflated after it has been extended from the endoscope.

Therefore, there is a long-felt need in the art for a system and method for utilizing an endoscopic disc removal tool which allows use of tools too large to fit through the cannula portion of the endoscope. The tools should at least include rasps, burrs, Curettes, Kerrison's, inserters, brushes trials and shavers. The tools should be manipulable from the distal end of the endoscope through the cannula.

Thus, the present invention provides a system and method for using tools with an endoscope during a surgical procedure that are too large to fit through the cannula of the endoscope.

### SUMMARY OF THE INVENTION

Briefly, the invention involves a system and method for utilizing tools larger than the cannula bore of an endoscope in an endoscopic procedure. The system includes an endoscope having a cannula with an open or ported distal end. The ported distal end allows the shaft of an elongated tool having an oversized functional distal end to extend through the port with the oversized end in front of the cannula bore. The tool and the endoscope cannula are placed through a dilator tube that extends from outside the patient to the surgical site. A removeable and re-attachable handle is securable to the shaft of the oversized tool, which allows the surgeon to manipulate the tool through the cannula while visualizing the procedure utilizing the endoscope during the procedure. This construction allows the surgeon to utilize tools that are too large to fit through the cannula of the endoscope, including curettes, rasps, burrs, trials, Kerrison's, shavers and inserters.

Accordingly, it is an objective of the present invention to provide a system for utilizing tools having a functional head that is larger than the cannula bore.

It is a further objective of the present invention to provide a method for utilizing tools having a functional head that is larger than the cannula bore.

It is yet a further objective of the present invention to provide an endoscopic surgical system that allows for using tools like curettes, rasps, burrs, trials, Kerrison's, shavers and inserters.

It is another objective of the present invention to provide an endoscope that includes a ported distal end, allowing a surgical tool to be manipulated through the endoscope with a handle while vision is maintained through the endoscope during tool use.

Other objectives and advantages of this invention will become apparent from the following description taken in conjunction with the accompanying drawings wherein are set forth, by way of illustration and example, certain embodiments of this invention. The drawings constitute a part of this specification, include exemplary embodiments of the present invention, and illustrate various objects and features thereof.

### BRIEF DESCRIPTION OF THE FIGURES

FIG. 1 is a top front left perspective view of a human spine, illustrating the connection of the bones and vertebrae;

FIG. 2 is a partial front perspective view of FIG. 1, illustrating the L1 through L5 portion of the spine of FIG. 1;

FIG. 3 is a left side view of the L3-L4 portion of the spine shown in FIG. 1;

FIG. 4 is a section view taken along lines 4-4 of FIG. 3, illustrating a disc and a vertebra;

FIG. 5 is a perspective view of the vertebrae illustrated in FIG. 4, further showing the disc and the vertebrae;

FIG. 6 is a partial section view taken along lines 6-6 of FIG. 5;

FIG. 7 is a side view of one embodiment of the endoscope and surgical tool suitable for use with the present system;

FIG. 8 is a partial view of the embodiment shown in FIG. 7, illustrated with the handle removed for manipulating the surgical tool;

FIG. 9 is an exploded view of the embodiment shown in FIG. 7;

FIG. 10 is a partial side view of the endoscopic system, illustrating the tool tube and the surgical tool;

FIG. 11 is a partial side view, illustrating insertion of a Jamshidi needle to the surgical site;

FIG. 12 is a partial side view, illustrating insertion of dilation tubes over the Jamshidi needle to open a channel to the surgical site;

FIG. 13 is a partial side view, illustrating insertion of further dilation tubes to expand the channel to the surgical site;

FIG. 14 is a partial side view, illustrating the tool tube in position, as well as the surgical tool through the endoscope; and

FIG. 15 is an illustrative view showing use of the present endoscopic system and method.

### DETAILED DESCRIPTION OF THE PREFERRED EMBODIMENTS

While the present invention is susceptible of embodiment in various forms, there is shown in the drawings and will hereinafter be described a presently preferred embodiment with the understanding that the present disclosure is to be considered an exemplification of the invention and is not intended to limit the invention to the specific embodiments illustrated. It should be appreciated that like drawing numbers on different drawing views identify identical, or functionally similar, structural elements of the invention.

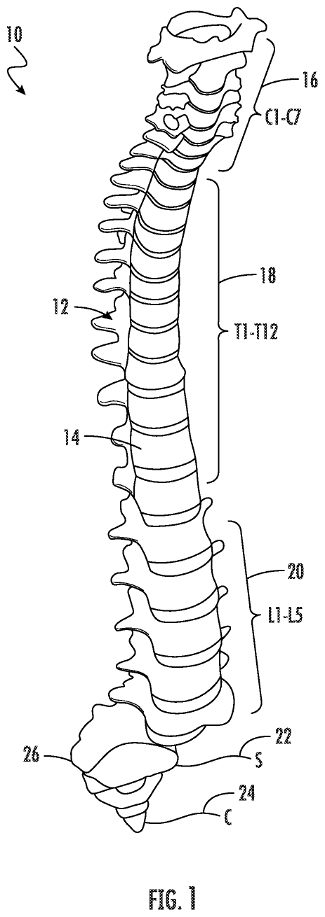

Referring generally to FIGS. 1-6, a human spinal column 10 is illustrated. As shown in FIG. 1, thirty-three individual bones 12 interlock with each other to form the spinal column 10. The vertebrae 14 are numbered and divided into regions. The cervical vertebrae 16, levels C1-C7, form the neck and support the head. The thoracic vertebrae 18, levels T1-T12, join with the ribs to form the rib cage (not shown). The five lumbar vertebrae 20, levels L1-L5, carry most of the weight of the upper body. Five vertebrae of the sacrum 22, level S, and four of the coccyx 24, level C, are fused to comprise the back wall of the pelvis 26.

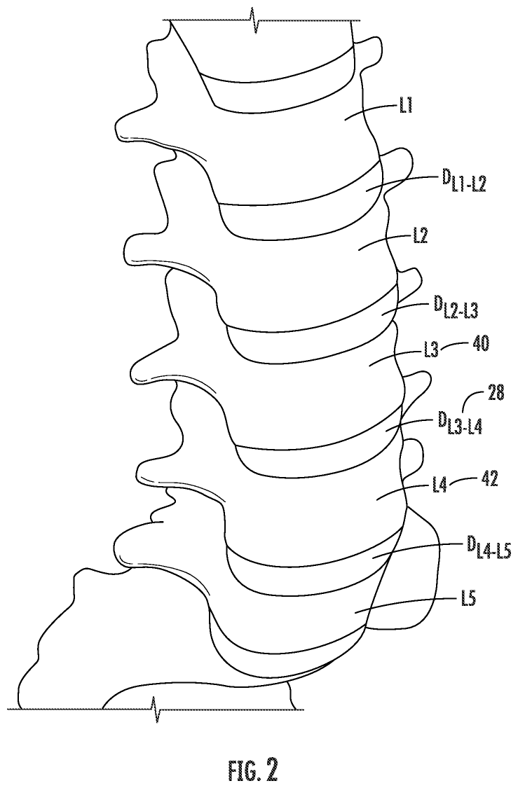

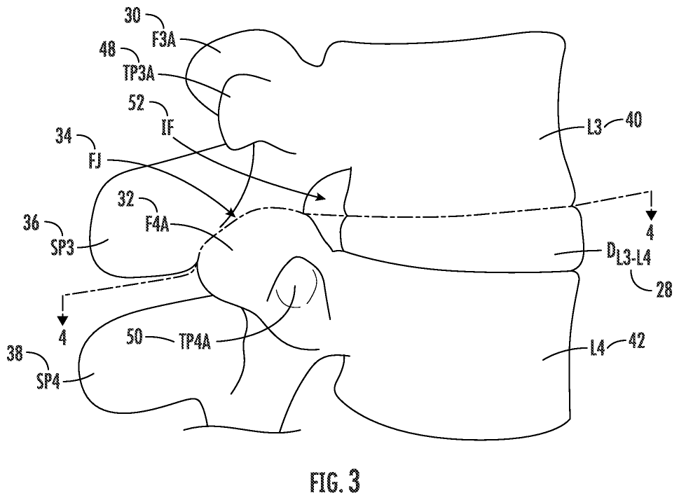

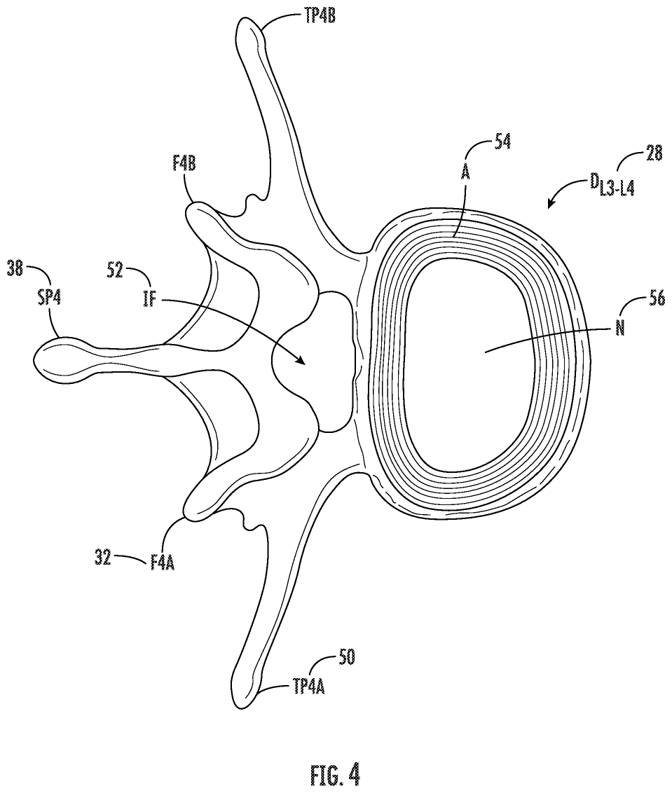

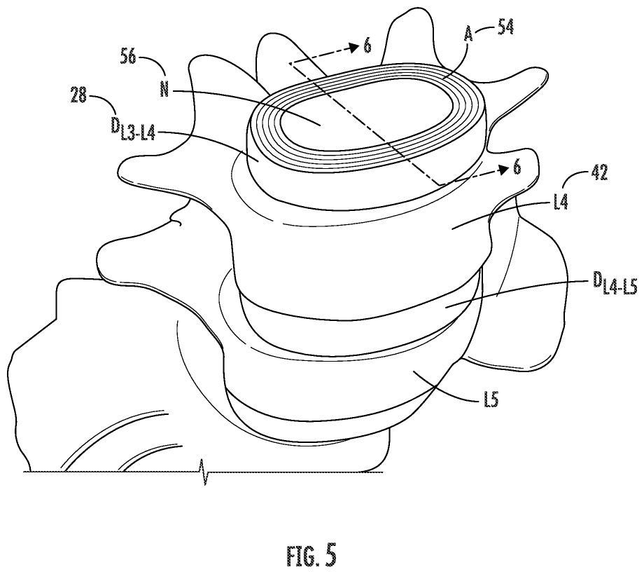

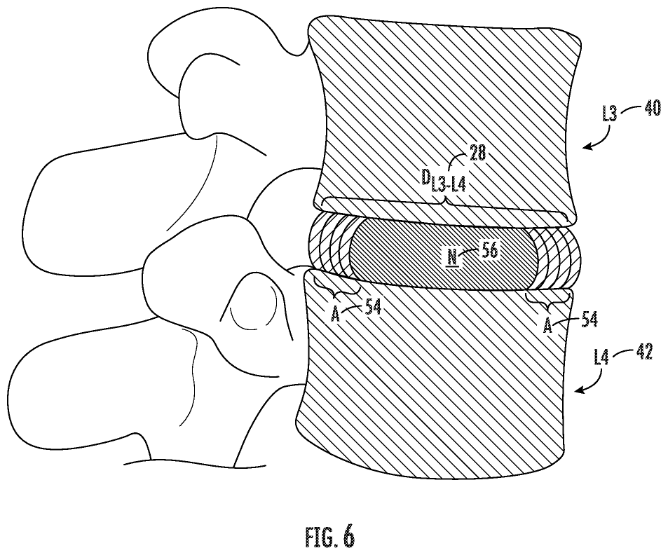

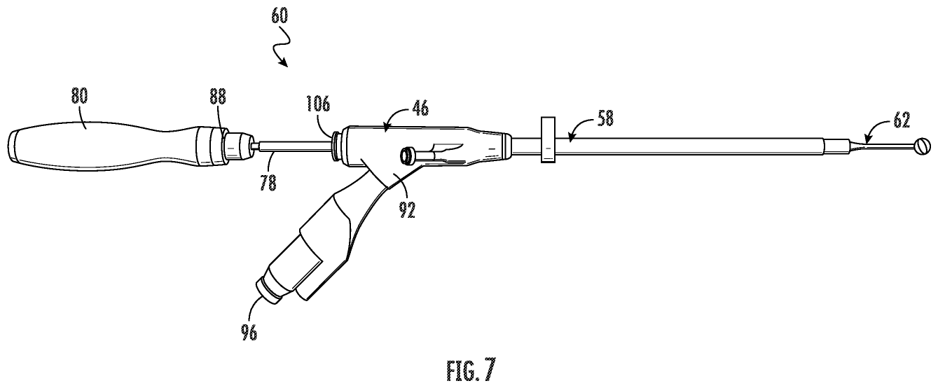

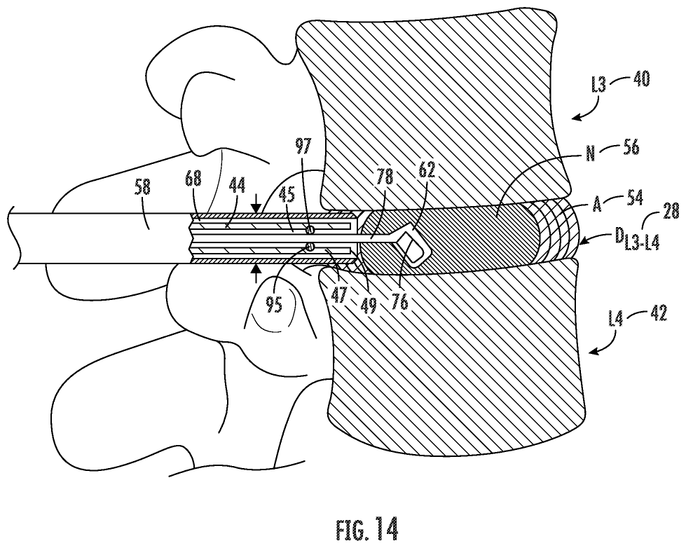

FIGS. 2-6 are illustrative of the removal of the nucleus of the disc between the L3 40 and L4 42 vertebrae, a common area of the spine for degeneration and pain in the spine where a surgeon may need to perform a discectomy. However, it should be noted that the teachings of the present system and method may be utilized along any portion of the spine for any procedure where a surgeon may need to utilize a tool that will not fit through the working channel 44 (FIG. 14) of an endoscope 46 (FIG. 7). FIG. 2 illustrates an anterior perspective view of the lumbar section 20 of the spinal column 10. Disc D.sub.L3-L4 28 is shown in an enlarged side view in FIG. 3. This figure also shows various anatomical structures of the spine, including facets F3A 30 and F4A 32, facet joint FJ 34, spinous processes SP3 36 and SP4 38, transverse processes TP3A 48 and TP4A 50, and intervertebral foramen IF 52. FIG. 4 is a top view of the section of the spinal column 10 shown in FIG. 3, with the L3 40 vertebra removed to expose annulus A 54 and nucleus N 56 of disc D.sub.L3-L4 28. FIG. 5 is an anterior perspective view of the section of the spinal column 10 shown in FIG. 4. FIG. 6 is a partial cross-sectional view of the section of the spinal column 10 shown in FIG. 5, but with vertebra L3 40 in place atop disc D.sub.L3-L4 28.

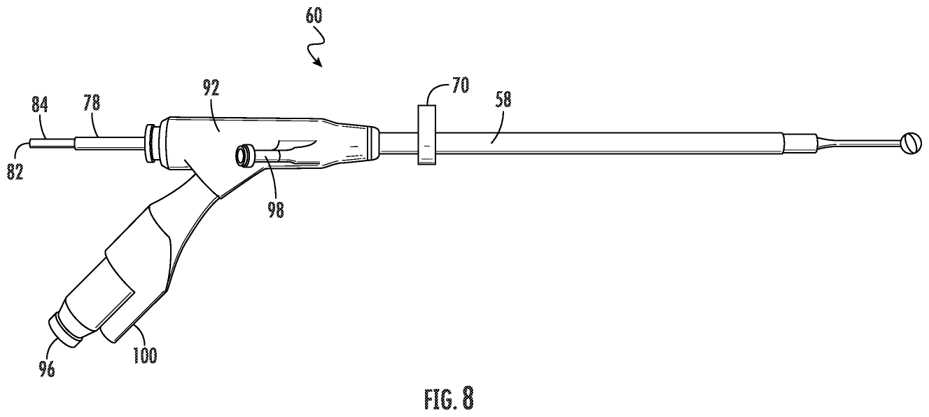

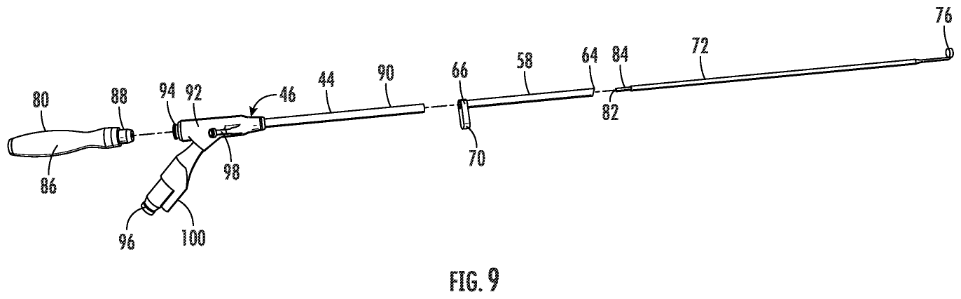

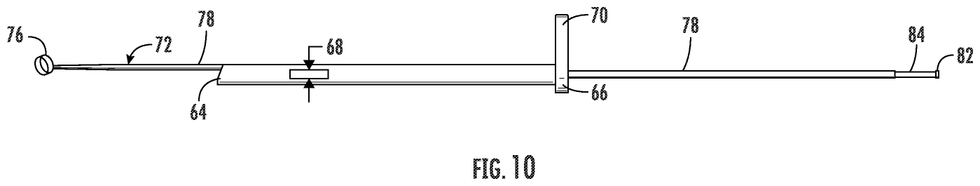

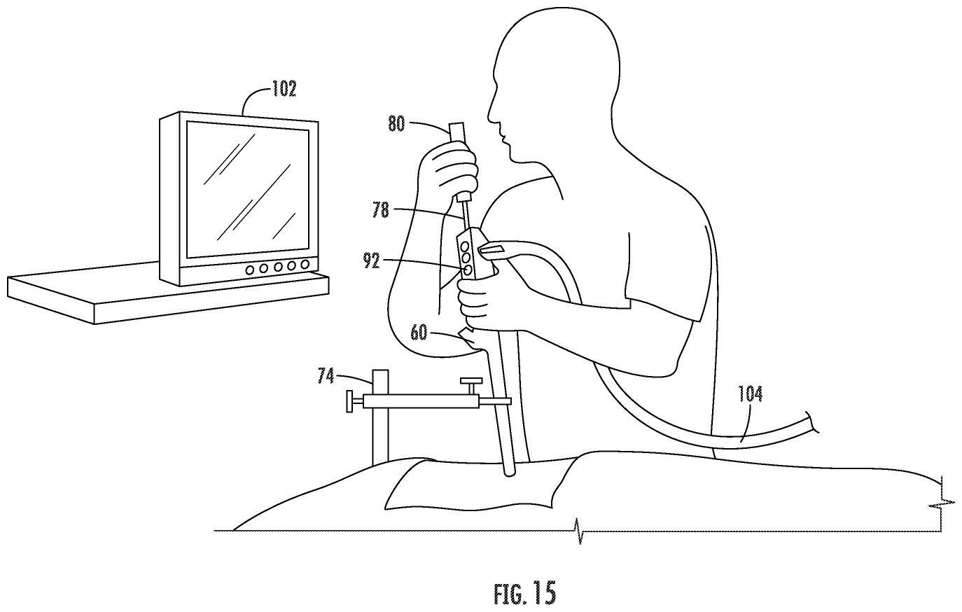

Referring to FIGS. 7-10, an endoscope assembly 60 constructed and arranged to carry out the present system and method is illustrated. The endoscope assembly 60 includes a tool tube 58, an endoscope 46, and an oversized tool assembly 62. The tool tube 58 includes a tool tube first end 64, a tool tube second end 66, and a stabilizer arm 70. An inner bore 68 extends through the tool tube 58 from the tool tube first end 64 to the tool tube second end 66. The tool tube inner bore 68 may be supplied having any sized bore that is suitable for the tools required to perform the operation. In this manner, the tool tube 58 may have a much larger diameter than is required for the passage of the endoscope working channel 44. When the present system and method is utilized with an implant inserter tool (not shown), the tool tube inner bore may be 1.2, 1.3, 1.4, 1.5, 1.6, 1.7, 1.8, 1.9, or even 2.0 times or more with respect to the size of the working channel 44 of the endoscope 46. The tool tube 58 has a sufficient length so that the tool tube second end 66 extends outside of the surgical patient, while the tool tube first end 64 is positioned adjacent to the surgical site to provide a pathway for the surgical tool 72 and the endoscope 46. The stabilizer arm 70 can be utilized to secure the tool tube 58 in a substantially fixed position during a surgical procedure. In one such example, the stabilizer arm 70 may be secured to the surgical table with a segmented mount assembly 74, (FIG. 15). It should be noted that the tool tube 58 can be utilized without the stabilizer arm 70 without departing from the scope of the invention. The surgical tool 72 includes a working end 76 that is sized to fit through the tool tube inner bore 68, but too large to fit through the lumen 45 of the working channel 44 of the endoscope 46 (FIG. 14). Tool working ends 76 may include, but should not be limited to, curettes, rasps, burrs, Kerrison's, inserters, brushes, trials, and shavers. A tool shaft 78 sized to fit through the lumen 45 of the working channel 44 of the endoscope 46 has sufficient length to extend completely through the endoscope 46 for attachment to a tool hand grip 80. The tool hand grip 80 may be secured to the distal end 82 of the tool shaft 78 in various manners suited to the best use of the tool working end 76 configuration. The securement configurations may include, but should not be limited to, bayonet mounts, threads, conjugate shapes 84 and the like. The securement configuration in a most preferred embodiment allows rotation as well as translation of the tool working end 76 through the lumen 45 of the working channel 44. The tool hand grip 80 includes a grip portion 86 sized for grasping with a human hand. The grip portion 86 may include rubber, serrations, or other grip enhancing surfaces that provide for securely grasping the hand grip portion with surgical gloves that may be wet. An attachment portion 88 is provided for securement to the distal end of the tool shaft 82 and is preferably constructed of metal to provide a rigid connection between the tool hand grip 80 and the distal end of the tool shaft 82. The endoscope 46 has a first end 90, the endoscope manifold 92, and the endoscope second end 94. The endoscope first end 90 includes the working channel 44; the working channel 44 having an inner lumen 45 of the working channel 44 that has a substantially constant diameter and shape extending from the endoscope first end 90 to the endoscope second end 94 through which endoscopic surgery is typically performed. The endoscope manifold 92 typically includes connections for video 96, pressurized fluid 100 which may be water or air, and a vacuum connection 98. Video may be provided by fiber optics 47 which extend through the lumen 45 of the working channel 44, or by a camera chip 49 that may be positioned near the first end 90 of the endoscope 46 within the lumen 45 of the working channel 44 with wiring extending through the lumen 45 of the working channel 44 to the video connection 96. The video connection 96 is preferably connected to a monitor 102 via wiring 104 (FIG. 15). The second end 94 of the endoscope 46 includes a port 106 sized to allow passage of the tool shaft 78. In a preferred embodiment, an o-ring 97, seal 95 or the like (not shown) is positioned in the port 106 to cooperate with an outer surface of the tool shaft 78 to reduce or eliminate leakage of fluids and/or vacuum through the lumen 45 of the working channel 44.

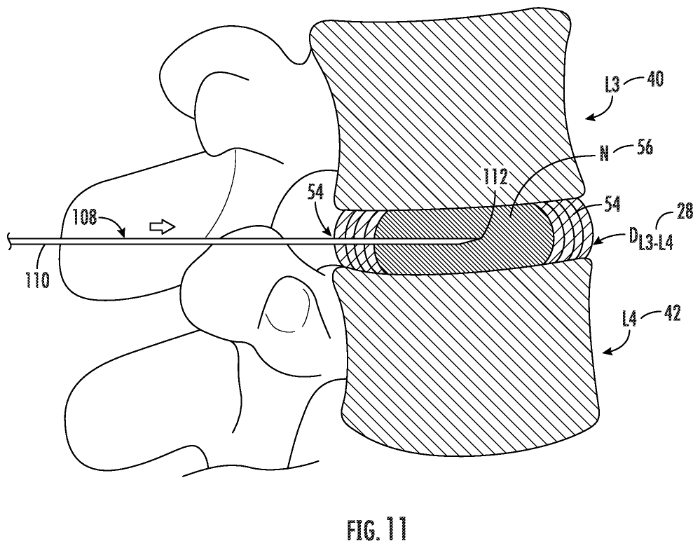

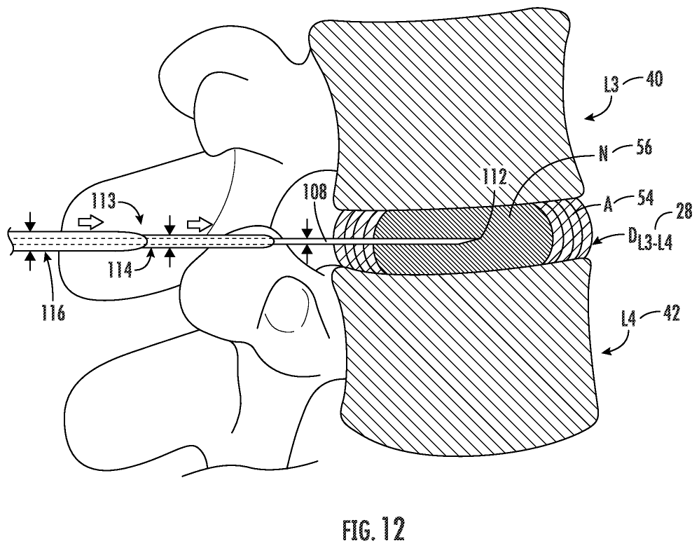

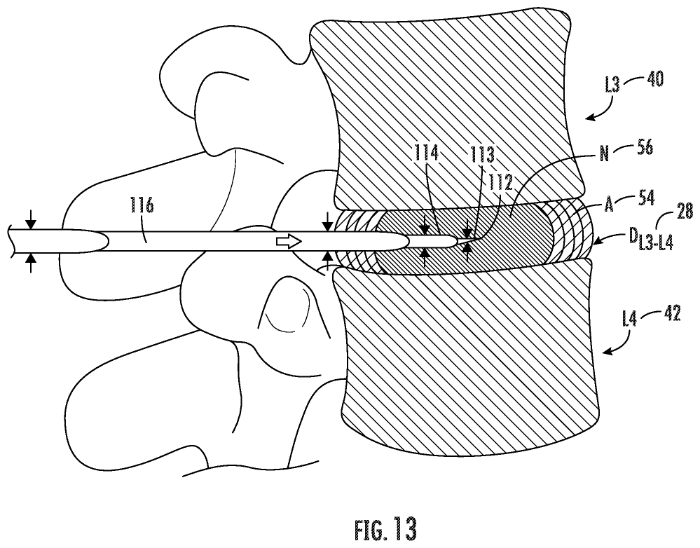

Referring generally to the figures, and more specifically to FIGS. 11-14, performance of a discectomy using the present system and method is illustrated. FIG. 11 illustrates a jamshidi needle 108 having a shaft portion 110 and point portion 112 piercing annulus 54 and nucleus 56 of disc 28 (D.sub.L3-L4). Once the surgeon has pierced the annulus 54 and entered the disc 28 (D.sub.L3-L4) with jamshidi needle 108, the entry channel is enlarged with a plurality of increasingly larger dilators 113, as best shown in FIG. 12. This figure shows the second dilator 114 and third dilator 116 being introduced down shaft portion 110, and eventually into annulus 54 and the center of nucleus 56 of disc 28 (D.sub.L3-L4). FIG. 13 further illustrates the discectomy (diskectomy) procedure, namely, introduction of progressively larger dilators 113, into the nucleus 56 of disc 28 (D.sub.L3-L4). This is done to increase the size of the opening in the annulus 54 and into the disc 28 for the eventual introduction of the tool tube 58 (shown in FIG. 14) proximate to the nucleus 56. Once the dilators 113 have made the opening large enough, the tool tube 58 is introduced over the dilators 113. FIG. 14 illustrates introduction of the tool tube 58 with its working channel 44 through annulus 54 of disc 28 (D.sub.L3-L4) proximate to nucleus 56 just prior to introduction of surgical instruments into the disc. When the tool tube 58 is in place proximate to nucleus 56, all dilators 113 are removed. The tool tube 58 may now be secured in position, if desired, with the stabilizer arm 70. A surgical tool 72 is now placed within the tool tube 58 with the working end 76 of the tool in close proximity to the disc 28. The tool shaft 78 is extending out of the tool tube 58 without the tool hand grip 80. The endoscope 46 is then threaded over the tool shaft 78 with the tool shaft extending through the lumen 45 of the working channel 44, and the outer diameter of the working channel 44 is simultaneously threaded into the lumen of the tool tube 58. Once the endoscope 46 is in position to view the surgery, the hand grip 80 is secured to the tool shaft 78. The video connection 96, pressurized fluid connection 100 and vacuum connection 98 may be attached prior to or after the placement of the endoscope 46 into the tool tube 58. In this manner, the surgeon can manipulate the working ends 76 of the surgical tools through the endoscope 46 while still maintaining a view of the surgical site with the endoscope 46. Should the surgeon need a different surgical tool 72, the hand grip 80 is removed from the surgical tool 72 and the endoscope 46 is removed from the tool tube 58. The tool 72 is replaced and the hand grip 80 may be attached prior to insertion of the endoscope 46 into the tool tube 58.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures, and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An endoscopic surgical system comprising:
an endoscope (46) having a first end (90) and a second end (94), the endoscope first end (90) including a working channel (44), the working channel (44) having an inner lumen (45) that has a substantially constant diameter and shape extending from the endoscope first end (90) to the endoscope second end (94);
a tool tube (58), including a tool tube first end (64), a tool tube second end (66), the tool tube (58) having an inner bore (68) extending through the tool tube (58) from the tool tube first end (64) to the tool tube second end (66), the tool tube (58) having a sufficient length to extend from a position adjacent a surgical site to outside of an anatomy;
a surgical tool (72) including a working end (76), a tool shaft (78) and a tool hand grip (80), wherein the tool shaft is sized to fit through the lumen (45) of the working channel (44) of the endoscope (46) having sufficient length to extend completely through the endoscope (46) for attachment to the tool hand grip (80), the working end (76) being sized to fit through the tool tube inner bore (68), but too large to fit through the lumen (45) of the working channel (44) of the endoscope (46), allowing rotation as well as linear translation of the tool working end (76) through the lumen (45) of the working channel (44).

2. The endoscopic surgical system of claim 1 wherein the endoscope (46) includes an endoscope manifold (92), the endoscope manifold (92) including at least one connection for video (96).

3. The endoscopic surgical system of claim 2 wherein the endoscope manifold (92) further includes a connection for pressurized fluid (100), wherein the pressurized fluid is directed to the first end (90) of the endoscope (46).

4. The endoscopic surgical system of claim 2 wherein the endoscope manifold (92) further includes a connection for vacuum (98).

5. The endoscopic surgical system of claim 2 wherein the endoscope (46) includes fiber optics (47) for viewing the area around the first end (90) of the endoscope (46).

6. The endoscopic surgical system of claim 2 wherein the endoscope (46) includes a camera chip (99) positioned near the first end (90) of the endoscope (46) within the lumen (45) of the working channel (44) with wiring extending through the lumen (45) of the working channel (44) to the video connection (96).

7. The endoscopic surgical system of claim 1 wherein the inner lumen (45) includes a seal (95), the seal (95) extending between an inner surface of the inner lumen (45) and an outer surface of the tool shaft (78).

8. The endoscopic surgical system of claim 7 wherein the seal (95) is an o-ring (97).

9. The endoscopic surgical system of claim 1 wherein the tool tube (58) includes a stabilizer arm (70) for securing the tool tube (58) rigidly in position.

10. The endoscopic surgical system of claim 1 wherein a distal end of the tool shaft (78) opposite the working end (76) includes an attachment portion (88) for removable attachment of the tool hand grip (80).

11. The endoscopic surgical system of claim 10 wherein the attachment portion (88) includes conjugate shapes (84) with respect to the tool hand grip (80) and the tool shaft (78) to prevent rotation therebetween and allow for the linear translation thereof.

12. An endoscopic surgical device comprising:
an endoscope (46) having a first end (90) and a second end (94), the endoscope first end (90) including a working channel (44), the working channel (44) having an inner lumen (45) that has a substantially constant diameter and shape extending from the endoscope first end (90) to the endoscope second end (94), an endoscope manifold (92), the endoscope manifold (92) including at least one connection for video (96) and a connection for pressurized fluid (100), wherein the pressurized fluid (100) is directed to the first end (90) of the endoscope (46);
a surgical tool (72) including a working end (76), a tool shaft (78) and a tool hand grip (80), wherein the tool shaft (78) is sized to fit through the lumen (45) of the working channel (44) of the endoscope (46) having sufficient length to extend completely through the endoscope (46) for attachment to the tool hand grip (80), the working end (76) being sized to fit through the tool tube inner bore (68), but too large to fit through the lumen (45) of the working channel (44) of the endoscope (46), allowing rotation as well as linear translation of the tool working end (76) through the lumen (45) of the working channel (44).

13. The endoscopic surgical device of claim 12 wherein the inner lumen (45) includes a seal, the seal (95) extending between an inner surface of the inner lumen (45) and an outer surface of the tool shaft (78).

14. The endoscopic surgical device of claim 13 wherein the seal (95) is an o-ring (97).

15. The endoscopic surgical device of claim 12 including a tool tube (58), the tool tube (58) including a tool tube first end (64), and a tool tube second end (66), the tool tube (58) having an inner bore (68) extending through the tool tube (58) from the tool tube first end (64) to the tool tube second end (66), the tool tube (58) having a sufficient length to extend from a position adjacent a surgical site to outside of an anatomy.
